(19) United States
(12) Patent Application Publication
(10) Pub. No.: US 2007/0167890 A1
(43) Pub. Date: Jul. 19, 2007

(54) Method for improving the appearance of the submandibular area of the human face

(76) Inventor: Sharon Kay Ford Metters, Pomona, CA (US)

Correspondence Address: LAW OFFICE OF MAI VU PARE, 9107 WILSHIRE BLVD. #450, BEVERLY HILLS, CA, 90210, US

(21) Appl. No.: 11/332,798

(22) Filed: Jan. 17, 2006

(51) Int. Cl. A61F 5/02

(52) U.S. Cl. 602/1

(57) ABSTRACT

The present invention relates to a method of improving the appearance of a submandibular area of a human face. The method is comprised of covering the submandibular area of a user with a band. Forming a tight seal between the submandibular area and the band, and increasing physical activity. Additionally, vitamin enriched emulsions known in the art may be applied to the user's submandibular area prior to covering the area with a band, for improved appearance and conditioning.

### FIELD OF THE INVENTION

The present invention relates to a method for improving the appearance of the submandibular region of the face.

### BACKGROUND OF THE INVENTION

There is a desire in cosmetology and fitness circles to reduce the size and appearance of the submandibular region of the human face. Prior art methods of achieving said goals include manual stimulation, topical chemical treatments, electric stimulation, as well as cosmetic surgery. These methods can be costly, inconvenient, ineffective or overly invasive and rely on expensive services from health and cosmetology professionals. The present invention is a method that is simple for the layperson to use and is more affordable and convenient than prior art methods.

It is an object of the present invention to provide a convenient, non-invasive method of minimizing the submandibular area of the face. It is also an object of the present invention to provide an inexpensive method of improving the appearance of the submandibular region.

### SUMMARY OF THE INVENTION

The present invention is a submandibular slimming method that works by raising the temperature of the submandibular area locally by placement of a band impervious or semi-impervious to moisture. The invention operates by raising the body temperature of the user and preventing the evaporation of sweat from the submandibular region for the purpose of providing targeted fat reduction. In a preferred embodiment of the invention, the band is placed around the submandibular region during cardiovascular exercise other physical activity (e.g.. housekeeping, gardening, etc.). However, physical movement of the submandibular area itself is not necessary for the method taught in the present invention to have effect.

The band is attached from the top of the users head to the submandibular region of the jaw. In a preferred embodiment of the invention, the band is secured in an area away from the chin so that a tight seal may be made between the user's submandibular region and the band. The practice of the invention requires a band like device, that is impervious to moisture and has the capability for adjustment.

The band may be constructed from a variety of materials that are impermeable or semi-permeable to water including, but not limited to neoprene, woven polyester, or nylon.

### BRIEF DESCRIPTION OF THE DRAWINGS

FIG. 1 shows the profile of a user wearing the slimming chin band.

FIG. 2 is a side planar view of the slimming chin band.

FIG. 3 is a top planar view of the slimming chin band.

FIGS. 4-6 shows different views of a user wearing the slimming chin band.

FIG. 7 shows a user applying emulsions to the submandibular area.

### DETAILED DESCRIPTION OF THE DRAWINGS

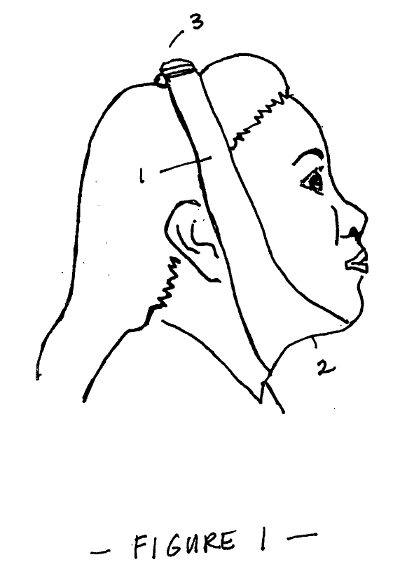

FIG. 1 shows a profile view of a user wearing a band 1 which is a key component of the present invention. As shown in FIG. 1, the band is wrapped around the submandibular area 2 of the user and secured up around the crown of the user's head 3. The invention can be secured at the crown of the user's head, as shown in FIG. 1.

FIG. 2 shows a side view of the band component of the present invention. The ends of the band 4 are used to secure the band to the submandibular area of the user and adjustment means 5 are provided so that the size of the band can be adjusted to accommodate the head size of various users.

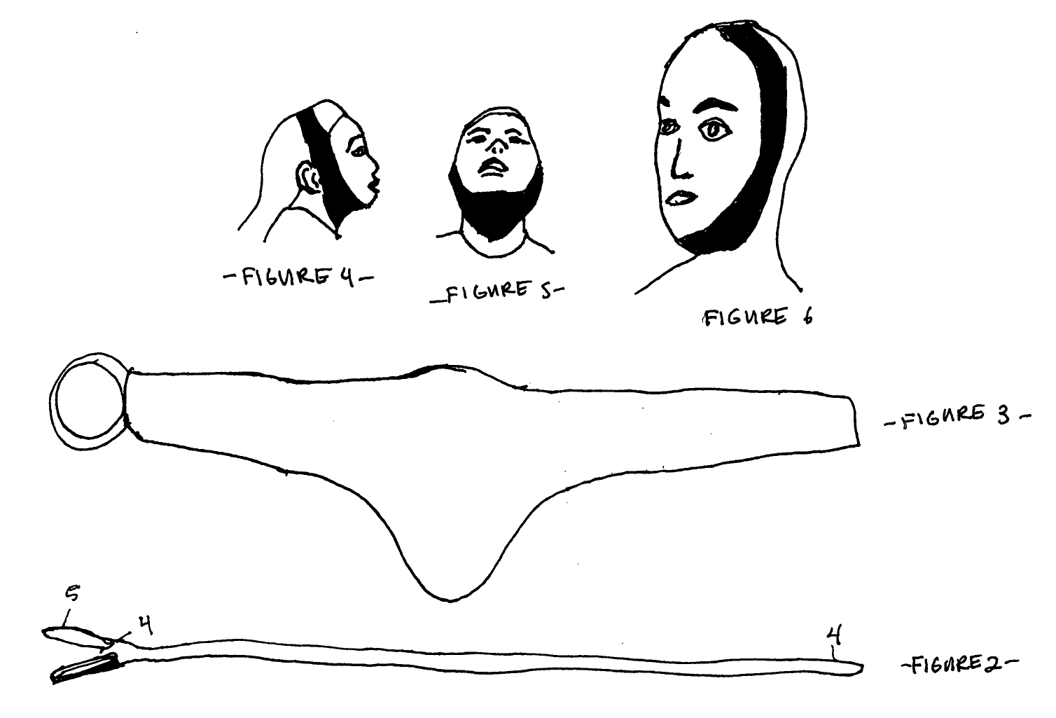

FIG. 3 shows a top planar view of the present invention. The area of material covering the submandibular region 6 is wide enough so that a large area of the submandibular area is covered, minimizing the amount of heat that can escape from this region. The present invention operates on the principle that preventing the evaporation of sweat from the submandibular region results in an increase in the amount of fluid lost in the area and minimizes the size and appearance of the region.

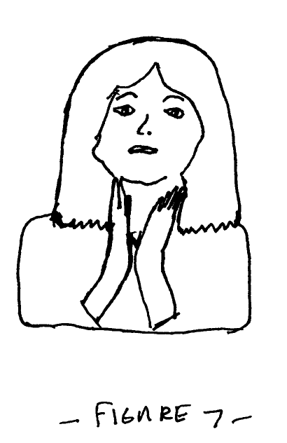

FIGS. 4-6 show various views of the user wearing the band component of the present invention. As can be seen in FIG. 5, the submandibular area of the user is fully covered.

FIG. 7 shows the user applying emulsions to the submandibular area prior to covering of the submandibular area with the band.

### DETAILED DESCRIPTION OF THE PRESENT INVENTION

The present invention is a method that comprises 1) raising the temperature of the submandibular region of the face through the use of a band which is impervious to moisture loss, 3) forming a tight seal between the submandibular area and the band 4) and increasing physical activity. In addition, before steps 1-3 above are performed, vitamin enriched emulsions may be applied to the submandibular region prior to placing the band over the submandibular area.

In raising the temperature of the submandibular region, a band having a thickness sufficient to cover the submandibular region of the user should be employed. As much of the submandibular area should be covered to improve the effectiveness of the present invention. By preventing the cooling of the submandibular area of the face from taking place, the temperature of the area is raised, promoting the additional loss of fluid from the area as the body attempts to cool down the submandibular area. In order to enhance heat production, the portion of the band covering the submandibular region must be constructed form material impermeable or semi-impermeable to moisture. Suitable materials include neoprene, wool, rubber, or plastic. In a preferred embodiment of the present invention, the band is constructed from neoprene.

Prior to fastening the band to the submandibular area of the user, lotions or emulsions known in the art can be applied to the submandibular region of the face. The lotions and emulsions known in the art contain one or more of the following vitamins: Vitamin A, Vitamin B, Vitamin C, Vitamin D, and/or Vitamin E. The emulsions can also include mineral oil, zinc oxide, dicalcium phosphate, gyceryl stearate, and/or wax. By applying the lotions and emulsions to the submandibular area prior to covering with the band, enhanced absorption of the ingredients may be allowed, providing improved appearance. After the emulsions or lotions are applied to the submandibular area of the face, the user places the band around the submandibular area of the face and securely fastens the band so that a tight seal is formed between the user's submandibular area and the band.

A final step in the present invention involves the user performing some level of physical activity. It is not necessary that the user actually physically move the submandibular area itself. It is only desirable if the user raises his overall body temperature by performing any sort of cardiovascular activity or heart rate but intense exercise is not mandatory in order for the present invention to have impact. Walking or performing light housework is sufficient.

Nor is it required that the user move the submandibular area of the face. The properties of the band prevent heat dissipation in the submandibular region of the user so that improvement in the appearance of the submandibular region occurs passively through the elimination of fluids in this area. While the present invention has little impact on a one-time or minimal use basis, regular practice of the inventive method improves the look and tone of the submandibular region.

With respect to the above description, it is to be realized that the optimum dimensional relationships for the parts of the invention, to include variations in size, materials, shape, form, function, manner and use are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A method to improve the appearance of a submandibular area of a human face comprising:
the placement of a band around the submandibular area of a user's face;
said band is impervious or semi-impervious to moisture loss;
forming a tight seal between the user's submandibular area and the band; and
increasing the user's cardiovascular activity.

2. A method to improve the appearance of a submandibular area of a human face comprising:
the placement of a vitamin enriched emulsion directly on the submandibular area of the user;
placement of a band around the submandibular area of a user's face;
said band is impervious or semi-impervious to moisture loss;
forming a tight seal between the user's submandibular area; and
increasing the user's cardiovascular level.
